(19) United States
(12) Patent Application Publication
(10) Pub. No.: US 2025/0334567 A1
(43) Pub. Date: Oct. 30, 2025

(54) DETERMINATION METHOD FOR PURITY AND POPULATION DOUBLING TIME OF HUMAN UMBILICAL CORD MESENCHYMAL STEM CELLS (hUC-MSCs)

(71) Applicant: SCLnow Biotechnology Co., Ltd., Beijing (CN)

(72) Inventors: Cheng Li, Beijing (CN); Lei Guo, Beijing (CN)

(21) Appl. No.: 18/742,935

(22) Filed: Jun. 13, 2024

(30) Foreign Application Priority Data
Apr. 30, 2024 (CN) 2024105413288

(51) Int. Cl. G01N 33/50; G01N 33/58; G01N 33/68

(52) U.S. Cl. CPC G01N 33/5005; G01N 33/582; G01N 33/6854

(57) ABSTRACT

The present invention relates to the technical field of stem cell detection, and particularly relates to a determination method for purity and population doubling time of human umbilical cord mesenchymal stem cells (hUC-MSCs). The cultured hUC-MSCs are mixed with the buffer solution to obtain cell suspension; the cell suspension is mixed and incubated with a fluorescein-labeled antibody to obtain an incubated substance; and the incubated substance is centrifuged, and the precipitate obtained is cleaned and then mixed with the buffer solution to obtain a substance to be tested. The present invention analyzes cell surface markers and cell cycles by using a flow cytometer and monoclonal antibodies labeled with different fluoresceins of hUC-MSCs as well as PI staining, and further defines a determination method for purity and population doubling time of hUC-MSCs.

### FIELD OF THE INVENTION

The present invention relates to the technical field of stem cell detection, and particularly relates to a determination method for purity and population doubling time of human umbilical cord mesenchymal stem cells (hUC-MSCs).

### BACKGROUND OF THE INVENTION

Mesenchymal stromal/stem cells (MSCs) are a kind of adult stem cells other than hematopoietic stem cells that exist in a variety of tissues (such as bone marrow, umbilical cord blood, umbilical cord tissue, placenta tissue and adipose tissue) and have a multi-directional differentiation potential. This kind of stem cells have a potential to differentiate into a variety of mesenchymal series cells (such as osteoblasts, chondroblasts and lipoblasts) or non-mesenchymal series cells, and have a unique cytokine secretion function.

Multipotency of MSCs means an ability to form more than one type of cells in organisms, which can induce differentiation into a variety of functional cells such as osteocytes and adipocytes in vitro, and can express genetic characteristics that regulate cell differentiation into other histiocyte types or organs (for example, the genes of SSEA-4, OCT-4, SOX-2 and NANOG are multipotent genes). MSCs have a characteristic of producing new cells by cell division, including a feature that the proportion of different stages in a cell cycle and the number of cells during cell division are changed over time. The purity and population doubling time of the hUC-MSCs need to be determined in order to control the quality requirements of MSCs.

### SUMMARY OF THE INVENTION

To solve the above problem, the present invention provides a determination method for purity and population doubling time of human umbilical cord mesenchymal stem cells (hUC-MSCs).

To achieve the above purpose, the present invention provides the following technical solution:

The present invention provides a determination method for purity and population doubling time of human umbilical cord mesenchymal stem cells (hUC-MSCs), comprising the following steps:
1) Mixing cultured hUC-MSCs with a buffer solution to obtain cell suspension;
2) Mixing and incubating the cell suspension obtained in step 1) with a fluorescein-labeled antibody to obtain an incubated substance;
3) Centrifuging the incubated substance obtained in step 2), cleaning the precipitate obtained and then mixing with the buffer solution to obtain a substance to be tested, and the substance to be tested is used to determine the positive expression ratio of cells;
4) Mixing the cultured hUC-MSCs with the buffer solution and an anhydrous ethanol solution to obtain an immobilized substance after immobilization;
5) Centrifuging the immobilized substance obtained in step 4), mixing, incubating and centrifuging the precipitate obtained with a PI working solution, cleaning the precipitate obtained, then mixing with the buffer solution, and analyzing population doubling time using Multicycle for DNA software;

No time sequence restriction exists between steps 1)-3) and steps 4)-5).

Preferably, the passage number of the hUC-MSCs is any of 3^{rd} to 6^{th} generations.

Preferably, the ratio of the number of the hUC-MSCs to the volume of the buffer solution is 1×10⁵:0.5 ml in step 1).

Preferably, the volume ratio of the cell suspension to the fluorescein-labeled antibody is 100:1 in step 2);

The antibody is one or more of anti-human CD11b, CD14, CD19, CD34, CD45, CD73, CD90, CD29, CD44, CD105, HLA-ABC and HLA-DR antibodies;

An FITC-labeled or PE-labeled antibody is used.

Preferably, the conditions of the incubation in step 2) include: the temperature is 4° C., the time is 30 min, and the incubation is conducted away from light.

Preferably, the conditions of the centrifugation include: the temperature is 4° C., the time is 5 min, and the revolving speed is 1200 rpm.

Preferably, the ratio of the number of the hUC-MSCs to the volume of the buffer solution to the volume of the anhydrous ethanol solution is 1×10⁵:1 ml: 3 ml in step 4), and the volume percent of the anhydrous ethanol solution is 75%.

Preferably, the conditions of the immobilization in step 4) include: the temperature is 4° C., and the time is 12-24 h.

Preferably, the final concentration of the PI working solution is 50 μg/ml in step 5);

The conditions of the incubation include: the temperature is 20-25° C., the time is 30 min, and the incubation is conducted away from light.

Preferably, the buffer solution is PBS buffer solution.

The present invention has the following beneficial effects:

The present invention analyzes cell surface markers and cell cycles by using a flow cytometer and monoclonal antibodies labeled with different fluoresceins of hUC-MSCs as well as PI staining, and further defines a determination method for purity and population doubling time of hUC-MSCs.

### DESCRIPTION OF DRAWINGS

To more clearly describe the technical solution in the embodiments of the present invention or in the prior art, the drawings required to be used in the embodiments will be simply presented below.

FIG. 1 is a flow diagram of labeling CD11b, CD14, CD19, CD34, CD45, CD73, CD90, CD29, CD44, CD105, HLA-ABC and HLA-DR antibodies with hUC-MSCs;

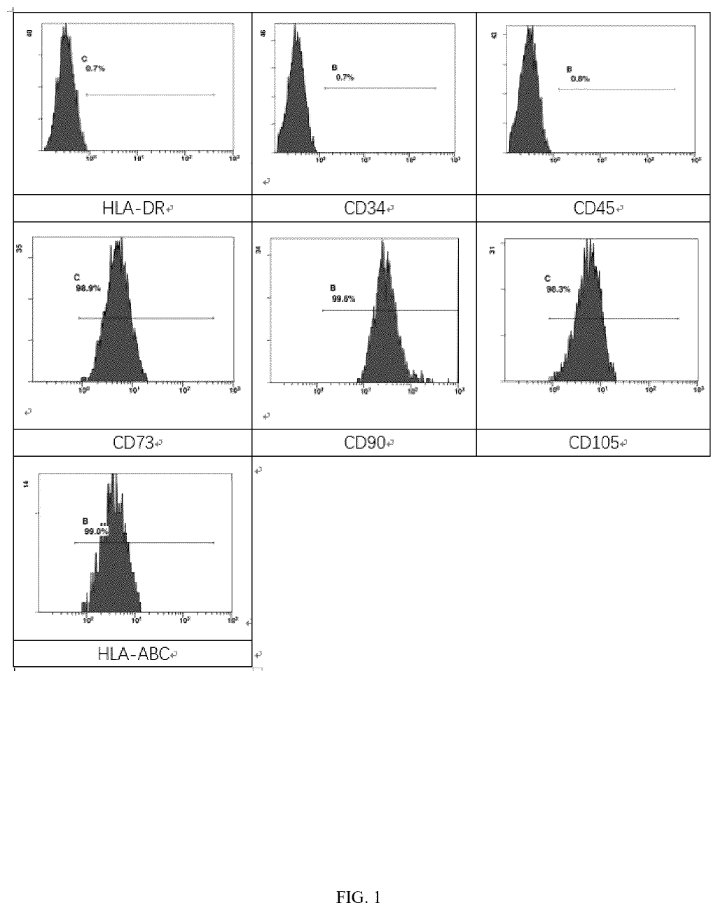

FIG. 2 is a diagram of a cell cycle obtained by hUC-MSCs through experimental steps of the present invention.

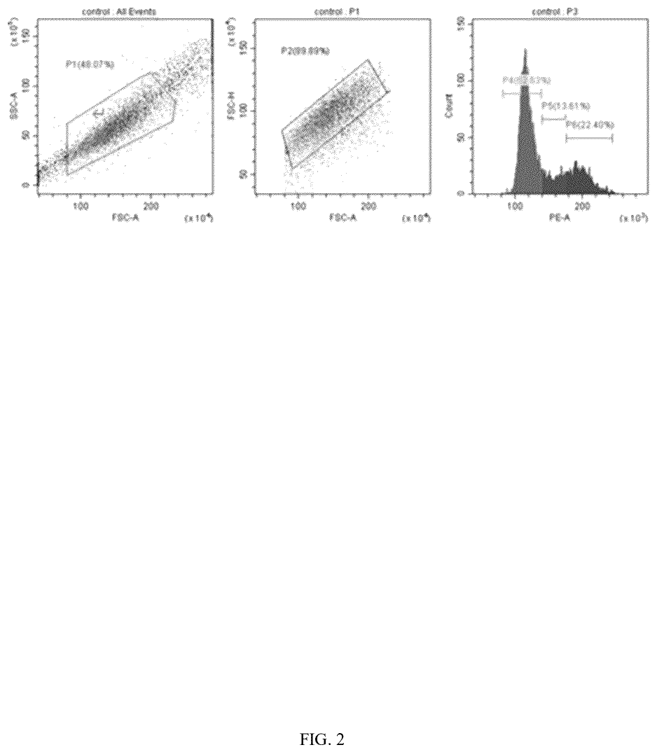

### DETAILED DESCRIPTION OF THE INVENTION

The present invention provides a determination method for purity and population doubling time of human umbilical cord mesenchymal stem cells (hUC-MSCs), comprising the following steps:
1) Mixing cultured hUC-MSCs with a buffer solution to obtain cell suspension;
2) Mixing and incubating the cell suspension obtained in step 1) with a fluorescein-labeled antibody to obtain an incubated substance;
3) Centrifuging the incubated substance obtained in step 2), cleaning the precipitate obtained and then mixing with the buffer solution to obtain a substance to be tested, and the substance to be tested is used to determine the positive expression ratio of cells;
4) Mixing the cultured hUC-MSCs with the buffer solution and an anhydrous ethanol solution to obtain an immobilized substance after immobilization;
5) Centrifuging the immobilized substance obtained in step 4), mixing, incubating and centrifuging the precipitate obtained with a PI working solution, cleaning the precipitate obtained, then mixing with the buffer solution, and analyzing population doubling time using Multicycle for DNA software.

No time sequence restriction exists between steps 1)-3) and steps 4)-5).

In the present invention, the cultured hUC-MSCs are mixed with the buffer solution to obtain cell suspension.

The present invention has no special restriction on methods for culturing hUC-MSCs, which can be obtained by those skilled in the art according to conventional culture methods. In the present invention, the passage number of the hUC-MSCs is preferably any of 3^{rd} to 6^{th} generations. In the present invention, the ratio of the number of the hUC-MSCs to the volume of the buffer solution is preferably 1×10⁵:0.5 ml. In the present invention, the buffer solution is preferably PBS buffer solution.

In the present invention, the cell suspension obtained is mixed and incubated with the fluorescein-labeled antibody to obtain an incubated substance.

In the present invention, the volume ratio of the cell suspension to the fluorescein-labeled antibody is preferably 100:1. In the present invention, the antibody is preferably one or more of anti-human CD11b, CD14, CD19, CD34, CD45, CD73, CD90, CD29, CD44, CD105, HLA-ABC and HLA-DR antibodies. In the present invention, an FITC-labeled or PE-labeled antibody is preferably used. In the present invention, the conditions of the incubation preferably include: the temperature is 4° C., the time is 30 min, and the incubation is conducted away from light.

In the present invention, the incubated substance obtained is centrifuged, the precipitate obtained is cleaned and then mixed with the buffer solution to obtain a substance to be tested, and the substance to be tested is used to determine the positive expression ratio of cells. In the present invention, the conditions of the centrifugation preferably include: the temperature is 4° C., the time is 5 min, and the revolving speed is 1200 rpm. In the present invention, the buffer solution is preferably PBS buffer solution.

In the present invention, the cultured hUC-MSCs are mixed with the buffer solution and the anhydrous ethanol solution to obtain an immobilized substance after immobilization. In the present invention, the ratio of the number of the hUC-MSCs to the volume of the buffer solution to the volume of the anhydrous ethanol solution is preferably 1×10⁵:1 ml: 3 ml. In the present invention, the volume percent of the anhydrous ethanol solution is preferably 75%. In the present invention, the conditions of the immobilization preferably include: the temperature is 4° C., and the time is 12-24 h.

In the present invention, the immobilized substance obtained is centrifuged, the precipitate obtained is mixed, incubated and centrifuged with the PI working solution, the precipitate obtained is cleaned and then mixed with the buffer solution, and the population doubling time is analyzed using Multicycle for DNA software. In the present invention, the final concentration of the PI working solution is 50 μg/ml. In the present invention, the conditions of the incubation preferably include: the temperature is 20-25° C., the time is 30 min, and the incubation is conducted away from light. In the present invention, the buffer solution is preferably PBS buffer solution.

To further describe the present invention, the prevent invention will be described below in detail in combination with embodiments, but the embodiments shall not be understood as the limitation of the protection scope of the present invention.

### Embodiment 1

### 1. Method and Principle

The positive expression ratio of cell surface markers is used to indicate cell purity. The cell surface markers as specific antigens can be bound with antibodies labeled with different fluoresceins (staining), single-cell suspension stained in a flow cytometer is pressed into a flow chamber by high pressure, cells are wrapped and pushed by sheath fluid into a single column and are ejected from the nozzle of the flow chamber at a rate of 5000-10000 cells per second, reflected light produced and fluorescent light emitted by the cells under the vertical irradiation of laser beams when flowing through a tested area successively are received by a receiver, all signals are converted into electrical pulse signals by photomultiplier tubes and then converted into digital signals that can be recognized by computers by analog/digital converters, and after processing and analysis, experimental results are reported in the form of histogram, two-dimensional point diagram, three-dimensional diagram, etc.

### 2. Reagents

Sodium chloride (NaCl): analytical reagent;

Sodium hydrogen phosphate (Na₂HPO₄·12H₂O): analytical reagent;

Potassium chloride (KCl): analytical reagent;

Potassium dihydrogen phosphate (KH₂PO₄): analytical reagent; 75% ethanol;

Propidium iodide (PI): percent ≥98%;

FITC-labeled or PE-labeled anti-human CD34, CD45, CD73, CD90, CD29, CD44, CD105, HLA-ABC and HLA-DR antibodies: with the sodium azide percent of ≤0.09%, suitable for testing with the flow cytometer;

An FITC-labeled or PE-labeled isotype control immunoglobulin: with the sodium azide percent of ≤0.09%, suitable for testing with the flow cytometer;

Calibration beads of the flow cytometer;

PBS buffer solution: 8.0 g of NaCl, 2.9 g of Na₂HPO₄·12H₂O, 0.2 g of KCl and 0.2 g of KH₂PO₄ are weighed respectively, dissolved with water, diluted to 1000 mL and subjected to autoclaved sterilization for later use after split charging.

### 3. Materials

A cell culture bottle: made of clinical-grade polypropylene material, with a breathable cover, sterile, non-pyrogenic and DNase/RNase free;

Centrifugal tubes, pipettes and tips: sterile, non-pyrogenic and DNase/RNase free;

A flow sample tube;

A 300-mesh sieve.

### 4. Instruments

Pipettes: with maximum ranges of 20 μL/1000 μL;

An electric continuous dispenser: suitable for 5 mL/10 mL pipettes;

A low speed freezing centrifuge: with a maximum centrifugal speed of 4500 r/min;

A flow cytometer;

A vortex oscillator;

A medicine refrigerator;

A PH meter: with measurement accuracy of +0.02.

A balance: with a reciprocal sensibility of 0.1 mg.

### 5. Testing

### 5.1 Instrument Self-Calibration

The calibration beads are taken out of the refrigerator, kept in equilibrium at room temperature for 10 min and fully mixed, and then 0.5 ml (15-20 drips) is taken and added to the flow sample tube; the sample tube is placed on an injection needle, the instrument conducts automatic acquisition, FS and SS voltages are adjusted so that the beads are located in the middle of FS/SS histograms, the beads in the figure are circled with an A gate, and the peak means in FL1, FL2 and each fluorescence histogram are adjusted to about 500; and after the conditions of the instruments are adjusted, 5000 beads are collected. The variation coefficients (HPX-CVs) of FS, SS and each fluorescence parameter histogram are examined: the HPX-CV value should be below 3%, the difference between FS recorded each time and the voltage value of each fluorescence parameter should be ±5%, sample testing can be conducted, and the testing result is reliable.

### 5.2 Determination of Sample Purity

### 5.2.1 Sample Preparation

1×10⁵ cells (P3 to P6 generations) are collected and added with 500 μL of PBS buffer solution to prepare cell suspension which is added to the flow sample tube; according to the instructions for antibodies (Biolegend), 5 ul of fluorescein-labeled antibody or isotype control is added to each tube of cell suspension, subjected to vortex blending and then incubated away from light in a 4° C. refrigerator for 30 min; and each tube of cells is taken out of the refrigerator and centrifuged at 1200 r/min at 4° C. for 5 min, supernatant is removed, 1 mL of PBS buffer solution is added for vortex oscillation and blending to clean residual antibodies, after repeated cleaning for 2 times, 500 μL of PBS buffer solution is added for resuspension and blending, screened through the 300-mesh sieve and then added to the flow sample tube, and on-machine testing is completed within 2 h.

### 5.2.2 Sample Testing

The FITC-labeled or PE-labeled isotype control sample tube is put on the injection needle, the instrument conducts automatic acquisition, the FS and SS voltages are adjusted one by one, and the A gate is adjusted to circle target cells so that the cells in the FS/SS point diagrams are distributed well. FL1 and FL2 voltages are adjusted so that the target cells appear in negative regions on the left sides of FL1 log and FL2 Log, and two channel positive regions (B gate and C gate) are set respectively.

The cell sample tube to be tested is put on the injection needle, the instrument automatically acquires 10000 cells, and the positive expression ratio of the cells is analyzed.

### 5.3 Determination of Sample Cell Cycle

### 5.3.1 Sample Preparation

1×10⁵ cells (P3 to P6 generations) are collected and added with 1 mL of PBS buffer solution to prepare cell suspension, and 4 mL of pre-cooled 75% anhydrous ethanol solution is slowly dripped onto the vortex oscillator and immobilized in a 4° C. medicine refrigerator for 12-24 h; after immobilization, the cells are collected by centrifuging at 1200 r/min at 4° C. for 5 min, and after slight vortex oscillation, 500 μL of 5 mg/ml PI solution is added for incubation away from light at room temperature for 30 min; and after incubation, centrifugation is conducted at 1200 r/min at 4° C. for 5 min, supernatant is removed, 1 mL of PBS buffer solution is added for vortex oscillation and blending to clean residual PI, after repeated cleaning for 2 times, 500 μL of PBS buffer solution is added for resuspension and blending, screened through the 300-mesh sieve and then added to the flow sample tube, and on-machine testing is completed within 2 h.

### 5.3.2 Sample Testing

The cell sample tube to be tested is put on the injection needle, the instrument automatically acquires 20000 cells, and the cell cycle is analyzed using Multicycle for DNA software.

### 5.3 Result Analysis

Purity: the percentage of positive cells labeled with corresponding antibodies in the total number of the tested cells.

Cell cycle: the Multicycle for DNA software conducts automatic fitting, the fitting result shows “Good”, and the percentage of cells at the G0/G1 phase is recorded.

The present invention is described in detail by the above embodiments. However, the above embodiments are merely part of the embodiments of the present invention, not all of the embodiments, and other embodiments can be obtained based on the embodiments in the present invention without contributing creative labor, which will also belong to the protection scope of the present invention.

What is claimed is:

1. A determination method for purity and population doubling time of human umbilical cord mesenchymal stem cells (hUC-MSCs), comprising the following steps:
1) mixing cultured hUC-MSCs with a buffer solution to obtain cell suspension;
2) mixing and incubating the cell suspension obtained in step 1) with a fluorescein-labeled antibody to obtain an incubated substance;
3) centrifuging the incubated substance obtained in step 2), cleaning the precipitate obtained and then mixing with the buffer solution to obtain a substance to be tested, and the substance to be tested is used to determine the positive expression ratio of cells;
4) mixing the cultured hUC-MSCs with the buffer solution and an anhydrous ethanol solution to obtain an immobilized substance after immobilization;
5) centrifuging the immobilized substance obtained in step 4), mixing, incubating and centrifuging the precipitate obtained with a PI working solution, cleaning the precipitate obtained, then mixing with the buffer solution, and analyzing population doubling time using Multicycle for DNA software;
no time sequence restriction exists between steps 1)-3) and steps 4)-5).

2. The determination method according to claim 1, wherein the passage number of the hUC-MSCs is any of 3^{rd} to 6^{th} generations.

3. The determination method according to claim 1, wherein the ratio of the number of the hUC-MSCs to the volume of the buffer solution is 1×10⁵:0.5 ml in step 1).

4. The determination method according to claim 1, wherein the volume ratio of the cell suspension to the fluorescein-labeled antibody is 100:1 in step 2);
the antibody is one or more of anti-human CD11b, CD14, CD19, CD34, CD45, CD73, CD90, CD29, CD44, CD105, HLA-ABC and HLA-DR antibodies;
an FITC-labeled or PE-labeled antibody is used.

5. The determination method according to claim 1, wherein the conditions of the incubation in step 2) include: the temperature is 4° C., the time is 30 min, and the incubation is conducted away from light.

6. The determination method according to claim 1, wherein the conditions of the centrifugation include: the temperature is 4° C., the time is 5 min, and the revolving speed is 1200 rpm.

7. The determination method according to claim 1, wherein the ratio of the number of the hUC-MSCs to the volume of the buffer solution to the volume of the anhydrous ethanol solution is 1×10⁵:1 ml: 3 ml in step 4), and the volume percent of the anhydrous ethanol solution is 75%.

8. The determination method according to claim 1, wherein the conditions of the immobilization in step 4) include: the temperature is 4° C., and the time is 12-24 hour.

9. The determination method according to claim 1, wherein the final concentration of the PI working solution is 50 μg/ml in step 5);
the conditions of the incubation include: the temperature is 20-25° C., the time is 30 min, and the incubation is conducted away from light.

10. The determination method according to claim 1, wherein the buffer solution is PBS buffer solution.
